(19) United States
(12) Patent Application Publication
(10) Pub. No.: US 2024/0255411 A1
(43) Pub. Date: Aug. 1, 2024

(54) PARTICLE MEASURING APPARATUS, ULTRAPURE WATER PRODUCTION APPARATUS HAVING THE SAME, AND PARTICLE MEASURING METHOD

(71) Applicant: ORGANO CORPORATION, Tokyo (JP)

(72) Inventors: Fumitaka ICHIHARA, Tokyo (JP); Hiroshi SUGAWARA, Tokyo (JP); Tsukasa KONDO, Tokyo (JP); Fumio SUDO, Tokyo (JP)

(73) Assignee: ORGANO CORPORATION, Tokyo (JP)

(21) Appl. No.: 18/565,684

(22) PCT Filed: Mar. 18, 2022
(86) PCT No.: PCT/JP2022/012519

§ 371 Date: Nov. 30, 2023

(30) Foreign Application Priority Data
Jun. 14, 2021 (JP) 2021-098602

(51) Int. Cl. G01N 15/1434; C02F 1/00; C02F 1/20; C02F 1/32; C02F 1/42; C02F 1/44; C02F 9/00; C02F 101/10; C02F 101/30; C02F 103/04; G01N 15/14; G01N 33/18

(52) U.S. Cl. CPC G01N 15/1434; C02F 1/008; C02F 9/00; G01N 33/18; C02F 1/20; C02F 1/32; C02F 1/42; C02F 2001/427; C02F 1/444; C02F 2101/10; C02F 2101/30; C02F 2103/04; C02F 2301/08; G01N 2015/1486

(57) ABSTRACT

A particle measuring apparatus is provided that has improved measurement accuracy regardless of particle diameters and that ensures prompt measurement. A particle measuring apparatus has: first and second particle counters that obtain a number of particles that are contained in water that flows in a predetermined section of an ultrapure water production apparatus; and particle number calculation means that calculates the number of the particles that are contained in the water that flows in the predetermined section for each particle diameter range based on measurement results of the first and second particle counters. The first particle counter and the second particle counter have different counting efficiencies.

### FIELD OF THE INVENTION

The present application is based on, and claims priority from, JP2021-098602, filed on Jun. 14, 2021, the disclosure of which is hereby incorporated by reference herein in its entirety.

The present invention relates to a particle measuring apparatus, an ultrapure water production apparatus having the same, and a particle measuring method, and particularly relates to an apparatus for measuring a particle number in ultrapure water that is produced by the ultrapure water production apparatus.

### DESCRIPTION OF THE RELATED ART

Recently, demand for the water quality of ultrapure water has become strong, and regarding particles in ultrapure water, both the reduction of concentration level and the stable management are being sought for smaller particles. The particle number in ultrapure water is measured by means of a light scattering liquid-borne particle counter (LPC) that uses scattered light that is emitted from particles to be measured when the particles are irradiated by a laser beam (WO2020/241476). Direct ophthalmoscopy is used to detect the diameters and the shapes of the particles as well as the particle number in ultrapure water (JP2016-55240). In direct microscopic counting, particles that are captured by a filter membrane are observed using an optical microscope, a scanning electron microscope or the like.

### SUMMARY OF THE INVENTION

An LPC radiates a laser beam that is condensed to an increased light density in a very limited region of a flow cell to detect fine particles having a range of very small diameters such as 50 nm or less. This type of LPC is called a partial counting and light scattering LPC. A partial counting and light scattering LPC entails large uncertainty in measuring the concentration of particles in that the light intensity that is detected varies even for particles of the same size depending on where the laser beam passes, and further, because particles are not detected where the laser beam does not pass. On the other hand, direct ophthalmoscopy uses a filter membrane having a smaller pore size than the diameters of particles to be measured and can accurately evaluate the particle number for each particle diameter range. However, obtaining a sample requires a lengthy filtering process, and the prompt detection of a change in the particle number in the ultrapure water is therefore difficult to obtain.

The present invention aims at providing a particle measuring apparatus that has improved measurement accuracy regardless of particle diameters and that ensures prompt measurement.

A particle measuring apparatus of the present invention comprises:
first and second particle counters that obtain a number of particles that are contained in water that flows in a predetermined section of an ultrapure water production apparatus; and
particle number calculation means that calculates the number of the particles that are contained in the water that flows in the predetermined section for each particle diameter range based on measurement results of the first and second particle counters, wherein
the first particle counter and the second particle counter have different counting efficiencies.

According to the present invention, it is possible to provide a particle measuring apparatus that has improved measurement accuracy regardless of particle diameters and that ensures prompt measurement.

The above and other objects, features, and advantages of the present invention will become apparent from the following description with reference to the accompanying drawings that illustrate examples of the present invention.

### BRIEF DESCRIPTION OF DRAWINGS

FIG. 1 is a schematic view of a subsystem of an ultrapure water production apparatus according to an embodiment of the present invention;

FIG. 2A is a view schematically illustrating the arrangement of a particle measuring apparatus;

FIG. 2B is a view schematically illustrating the arrangement of a particle measuring apparatus according to a modification;

FIG. 3 is a schematic view of the system used in the examples;

FIG. 4 is a graph showing the measurement results by the particle counters at measurement point P1;

FIG. 5A is a graph showing the measurement results by a particle counter at measurement point P1;

FIG. 5B is a graph showing the measurement results by a particle counter at measurement point P1;

FIG. 5C is a graph showing the measurement results by a particle counter at measurement point P1;

FIG. 6 is a graph showing the measurement results by the particle counters at measurement point P2;

FIG. 7A is a graph showing the measurement results by a particle counter at measurement point P2;

FIG. 7B is a graph showing the measurement results by a particle counter at measurement point P2;

FIG. 7C is a graph showing the measurement results by a particle counter at measurement point P2;

FIG. 8 is a graph showing the measurement results by the particle counters at measurement point P3;

FIG. 9A is a graph showing the measurement results by a particle counter at measurement point P3;

FIG. 9B is a graph showing the measurement results by a particle counter at measurement point P3; and

FIG. 9C is a graph showing the measurement results by a particle counter at measurement point P3.

### DESCRIPTION OF EMBODIMENTS

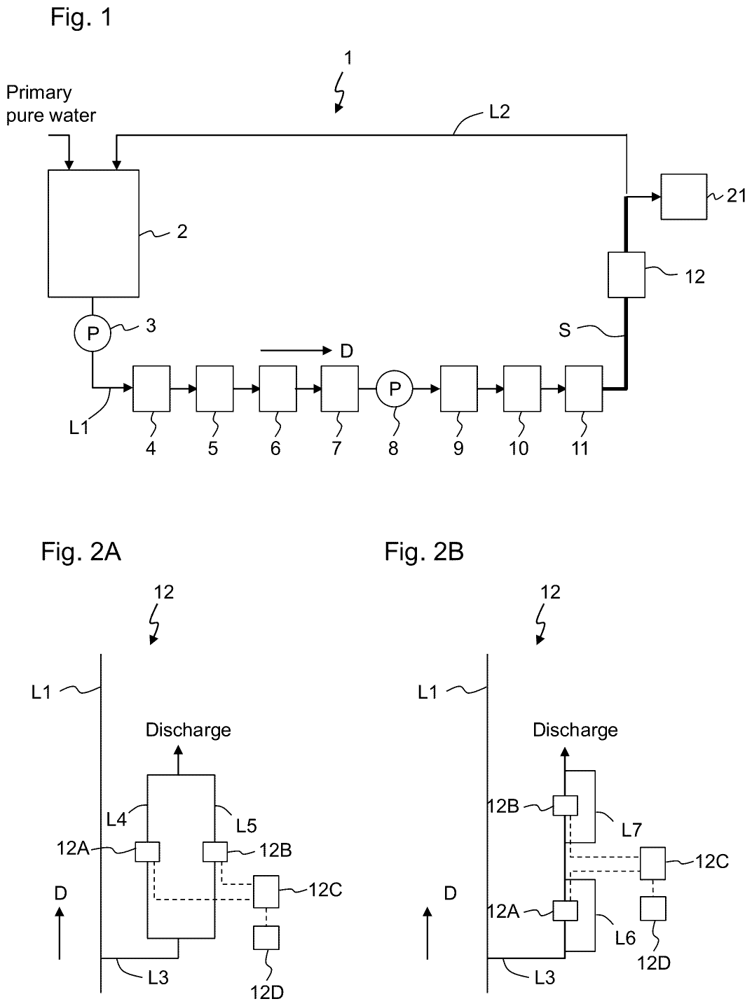

An embodiment of the present invention will be described with reference to the drawings. FIG. 1 schematically shows subsystem 1 of an ultrapure water production apparatus according to an embodiment of the present invention. Subsystem 1 is a system for producing ultrapure water from pure water that is produced by a primary pure water system and is also called a secondary pure water system. The ultrapure water is supplied to point of use 21. Subsystem 1 has primary pure water tank 2, pure water supply pump 3, ultraviolet ray oxidization apparatus 4, hydrogen peroxide removing apparatus 5, nonregenerative first ion exchange apparatus 6 of a mixed-bed type (a cartridge polisher), membrane deaerator apparatus 7, booster pump 8, second ion exchange apparatus 9, ultrafiltration membrane apparatus 10, and final-stage filter membrane apparatus 11, and these apparatuses are arranged in a series along main line L1 in the order listed above in flow direction D of the water to be treated. The branching point of main line L1 at which the line to point of use 21 branches is connected to primary pure water tank 2 by return line L2 that recirculates ultrapure water that has not been used at point of use 21 to primary pure water tank 2. Primary pure water tank 2 stores pure water that is produced by the primary pure water system.

Ultraviolet ray oxidization apparatus 4 radiates ultraviolet rays to the water to be treated to decompose organic materials that are contained in the water to be treated. Hydrogen peroxide removing apparatus 5 is provided with a catalyst such as palladium (Pd) or platinum (Pt) and decomposes hydrogen peroxide that is generated by the radiation of the ultraviolet rays. Damage to downstream first ion exchange apparatus 6 by oxidizing materials is thus prevented. First ion exchange apparatus 6 is charged with cation exchange resin and anion exchange resin in a mixed bed and removes ionic components in the water to be treated. Membrane deaerator apparatus 7 removes dissolved oxygen and carbon dioxide that are contained in the water to be treated. Booster pump 8 is provided to pressurize the water to be treated when, for example, point of use 21 is provided at a higher level. Second ion exchange apparatus 9 mainly removes particles or particulate matters that are generated by booster pump 8. Since particle and particulate matters can also be removed by ultrafiltration membrane apparatus 10, second ion exchange apparatus 9 may be omitted.

Ultrafiltration membrane apparatus 10 may use a membrane having a fractionation molecular cutoff of about 4000 to 6000 (equivalent to pore sizes of 2-4 nm) and particles having particle diameters of 10 nm or more can be removed highly efficiently. The membrane may be a hollow fiber membrane, a flat membrane, or a pleated type. Ultrafiltration membrane apparatus 10 may be, as in final-stage filter membrane apparatus 11, a piping that is charged with a filter membrane or a tower in which more than one cartridge are mounted. The ultrafiltration membrane is preferably a membrane having limited elution from the membrane itself, and polysulfone is preferably used. Examples of ultrafiltration membranes include OLT-6036H manufactured by Asahi Kasei Corporation and NTU-3306-K6R manufactured by Nitto Denko Corporation. Organic materials such as those eluted from the resin of first ion exchange apparatus 6 are removed by ultrafiltration membrane apparatus 10. Thus, the water quality of the ultrapure water that is supplied to point of use 21 is further improved and the load on final stage filter membrane apparatus 11 is reduced.

Final-stage filter membrane apparatus 11 is a purification unit that is provided at the final stage of subsystem 1. The filter membrane of final-stage filter membrane apparatus 11 is formed of materials such as polyethylene (PE), high density polyethylene (HDPE), polytetrafluoroethylene (PTFE), polypropylene (PP), polyarylsulfone (PAS), or nylon. The membrane may be a hollow fiber membrane, a flat membrane, or a pleated type. Final-stage filter membrane apparatus 11 is an apparatus in which cartridges of membranes are mounted in a housing. Alternatively, a piping that is charged with a filter membrane may be used as final-stage filter membrane apparatus 11. The piping is preferably formed of, for example, polyvinylidene difluoride (PVDF), PTFE, CLVP (clean polyvinyl chloride pipe), or perfluoroalkoxy alkane (PFA). As another alternative, a tower in which more than one cartridge is mounted may be used for final-stage filter membrane apparatus 11.

The retaining diameter of the filter membrane of final-stage filter membrane apparatus 11 is 5 nm or less, preferably 3 nm or less, and more preferably 1 nm or less. The retaining diameter is measured as follows: First, the particle removal efficiency (PRE) of the filter membrane to be measured is measured according to SEMI (Semiconductor Equipment and Materials International) Standard C89-0116 “TEST METHOD FOR PARTICLE REMOVAL PERFORMANCE OF LIQUID FILTER RATED BELOW 30 nm WITH INDUCTIVELY COUPLED PLASMA-MASS SPECTROSCOPY (ICP-MS)”. The retaining diameter means a particle diameter that can ensure achieves a PRE of 80% or more and preferably 90%, i.e., a particle diameter that can be captured at a probability of at least 80%. Therefore, a retaining diameter of 5 nm means that particles having particle diameters of 5 nm can be captured with a probability of 80% or more and preferably 90% and indicates a filtering performance having a rejection rate of 80% or more and preferably 90%. The filter membrane may be, for example, a Guardian (registered trademark) PS filter, manufactured by Entegris.

Final-stage filter membrane apparatus 11 is connected to point of use 21. Final-stage filter membrane apparatus 11 is the most downstream membrane filtering apparatus that constitutes the ultrapure water production apparatus. In subsystem 1, the ultrapure water that is taken from final-stage filter membrane apparatus 11 is supplied to point of use 21. The description “most downstream” means that, of the various purification units that constitute subsystem 1, this unit is at the most downstream side in flow direction D of the water to be treated.

One of either ultrafiltration membrane apparatus 10 or final-stage filter membrane apparatus 11 may be omitted. When final-stage filter membrane apparatus 11 is omitted, ultrafiltration membrane apparatus 10 becomes the most downstream membrane filtering apparatus that constitutes the ultrapure water production apparatus.

The ultrapure water production apparatus (subsystem 1) has particle measuring apparatus 12. Particle measuring apparatus 12 is provided in section S (the section shown by the bold line in FIG. 1) between final-stage filter membrane apparatus 11 and point of use 21. Final-stage filter membrane apparatus 11 is the most downstream membrane filtering apparatus that constitutes the ultrapure water production apparatus (when final-stage filter membrane apparatus 11 is omitted, the most downstream membrane filtering apparatus is ultrafiltration membrane apparatus 10). FIG. 2A schematically illustrates the arrangement of particle measuring apparatus 12. Particle measuring apparatus 12 includes first particle counter 12A and second particle counter 12B. First particle counter 12A and second particle counter 12B measure the number of particles that are contained in the water that flows in section S. First particle counter 12A and second particle counter 12B are laser beam scattering particle counters (LPCs). The LPCs radiate laser beams upon the particles to be detected, converts the scattered light that is emitted from the particles due to the irradiation of the laser beam to electric signals, and measures the particle number and the particle diameters based on the electric signals. Branch line L3 branches from main line L1 and branches out into two branch lines L4 and L5 that extend in parallel. First particle counter 12A is provided on branch line L4 and second particle counter 12B is provided on branch line L5. Therefore, first particle counter 12A and second particle counter 12B are provided at substantially equivalent points and equivalent ultrapure water is introduced to first particle counter 12A and second particle counter 12B. As will be described later in detail, since second particle counter 12B has a smaller rated flow rate than first particle counter 12A, valves for adjusting flow rates (not illustrated) are provided on branch lines L4 and L5. Alternatively, the diameters and the lengths of branch lines L4 and L5 may be determined in advance to obtain the rated flow rates of first particle counter 12A and second particle counter 12B. The ultrapure water that flows through first particle counter 12A and second particle counter 12B is discharged to outside the system but may be returned to main line L1.

“Substantially equivalent points” mean points within a section in which the particle numbers do not vary. As long as first particle counter 12A and second particle counter 12B are provided in such a section, these particle counters are considered to be arranged at substantially the same points even if they are remote from each other. For example, when first particle counter 12A and second particle counter 12B are provided in a section between final-stage filter membrane apparatus 11 and point of use 21 and the particle numbers do not vary, the particle counters are considered to be arranged at substantially equivalent points even if they are remote from each other. As will be described later, the same applies when first particle counter 12A and second particle counter 12B are provided in other sections. In this case, “substantially equivalent points” means any two positions in a section between two water treatment means that are arranged in a series without any other water treatment means interposed therebetween. For example, first particle counter 12A and second particle counter 12B are considered to be arranged at substantially equivalent positions if first particle counter 12A and second particle counter 12B are provided at any two positions in each section of the section between ultraviolet ray oxidization apparatus 4 and hydrogen peroxide removing apparatus 5, the section between first ion exchange apparatus 6 and membrane deaerator apparatus 7, the section between membrane deaerator apparatus 7 and second ion exchange apparatus 9, and the section between ultrafiltration membrane apparatus 10 and final-stage filter membrane apparatus 11 and the particle numbers do not vary.

As shown in FIG. 2B, first particle counter 12A and second particle counter 12B may be arranged in a series on branch line L3. In FIG. 2B, first particle counter 12A is arranged upstream of second particle counter 12B, but either of first particle counter 12A and second particle counter 12B may be arranged on the upstream side. Bypass line L6 that bypasses first particle counter 12A and bypass line L7 that bypasses second particle counter 12B are provided on branch line L3 to adjust the flow rates of the ultrapure water that is introduced to first particle counter 12A and second particle counter 12B. By combining first particle counter 12A with bypass line L6 and combining second particle counter 12B with bypass line L7, first particle counter 12A and second particle counter 12B having different rated flow rates can be arranged in a series. In addition, the reliability of the measurement is further improved because the same ultrapure water is introduced to first particle counter 12A and second particle counter 12B.

Particle measuring apparatus 12 is provided with particle number calculation means 12C that is connected to first particle counter 12A and second particle counter 12B. Calculation means 12C is provided as a personal computer or a control unit of the subsystem and is substantially constructed as software. Calculation means 12C calculates the number of particles that are contained in the water that flows in section S for each particle diameter range based on the measurements of first particle counter 12A and second particle counter 12B. The calculation method will be later described in detail.

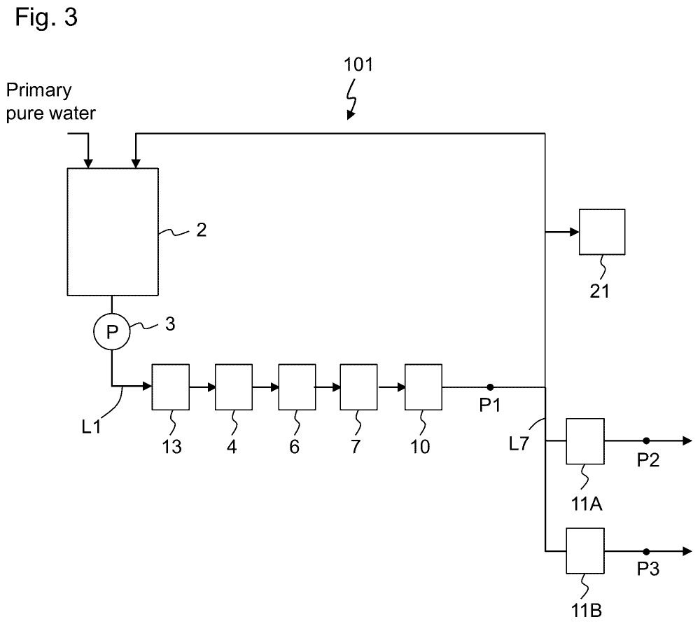

Examples will next be described. The particle number in ultrapure water was measured using system 101 shown in FIG. 3. System 101 that was used is a simplified version of subsystem 1 shown in FIG. 1 and is provided with heat exchanger 13 for adjusting the water temperature of the water to be treated between pure water supply pump 3 and ultraviolet ray oxidization apparatus 4. The most downstream membrane filtering apparatus that constitutes the ultrapure water production apparatus is ultrafiltration membrane apparatus 10, and filter membrane apparatuses 11A and 11B having the same filtering performance as final-stage filter membrane apparatus 11 are provided on branch line L7 that branches from main line L1 between ultrafiltration membrane apparatus 10 and point of use 21. Ultrafiltration membrane apparatus 10 is OLT-6036HA manufactured by Asahi Kasei Corporation, filter membrane apparatus 11A is Guardian (registered trademark) PS filter manufactured by Entegris (having a retaining diameter of 5 nm), and filter membrane apparatus 11B is Guardian (registered trademark) PS filter manufactured by Entegris (having a retaining diameter of 1 nm).

The particle number in the ultrapure water was measured at measurement points P1 to P3 shown in the figure. The particle number was measured by two particle counters (A) and (B) at each of measurement points P1 to P3. Particle counter (A) is Ultra DI-20 (manufactured by PMS) that can measure particles of 20 nm or more. Particle counter (B) is KS-16 (manufactured by RION) that can measure particles of 100 nm or more. In order to obtain reference values, the ultrapure water was sampled at measurement points P1 and P3 and was analyzed by a scanning electron microscope (SEM). Specifically, the ultrapure water was introduced to a centrifugal separator having a filter membrane, and the particles captured by the filter membrane were observed by the SEM to obtain the particle number for each particle diameter range (hereinafter, referred to as the SEM method). Particle counter (A) and particle counter (B) were arranged in parallel, as shown in FIG. 2A. The specifications of particle counters (A) and (B) and the SEM method are shown in Table 1.

TABLE 1

| | Ultra DI-20 | KS-16 | |
|---|---|---|---|
| | (particle | (particle | (SEM |
| Type | counter (A)) | counter (B)) | method) |
| Minimum measurable particle | 20 | 100 | 10 |
| diameter (nm) | | | |
| Particle diameter range (nm) | 20/50/75/100 | 100/150/200/ | (10/50/100) |
| | | 300/500 | |
| Rated flow rate (mL/min) | 75 | 10 | — |
| Net flow rate (mL/min) | 0.45 | 7 | — |

In the table, the particle diameter ranges of particle counters (A) and (B) indicate the measurement ranges. For example, particle counter (A) can simultaneously measure the numbers of particles having diameters of 20 nm or more, 50 nm or more, 75 nm or more, and 100 nm or more. The rated flow rate indicates the flow rate of the ultrapure water that is introduced to the particle counter. The net flow rate indicates the flow rate that contributes to the measurement of the particle number. Specifically, the net flow rate is the flow rate of a portion of the ultrapure water introduced to the particle counter to which a laser beam is radiated to measure the particle number, or the volume of a portion to which the laser beam is radiated per unit time to measure the particle number. The scattering intensity of the laser beam is proportional to the sixth power of the particle diameter, and particle counter (A) having a smaller minimum measurable particle diameter therefore requires a narrowed laser beam to radiate a strong laser beam upon a very limited region. As a result, most of the ultrapure water that is introduced to the particle counter is not irradiated by the laser beam and does not contribute to the measurement. Here, counting efficiency is defined as the net flow rate/the rated flow rate×100(%). Particle counter (A) has a significantly lower counting efficiency. On the other hand, particle counter (B) radiates a weaker laser beam than particle counter (A) upon a wider region. Thus, a large portion of the ultrapure water that is introduced to the particle counter contributes to the measurement and particle counter (B) has a higher counting efficiency.

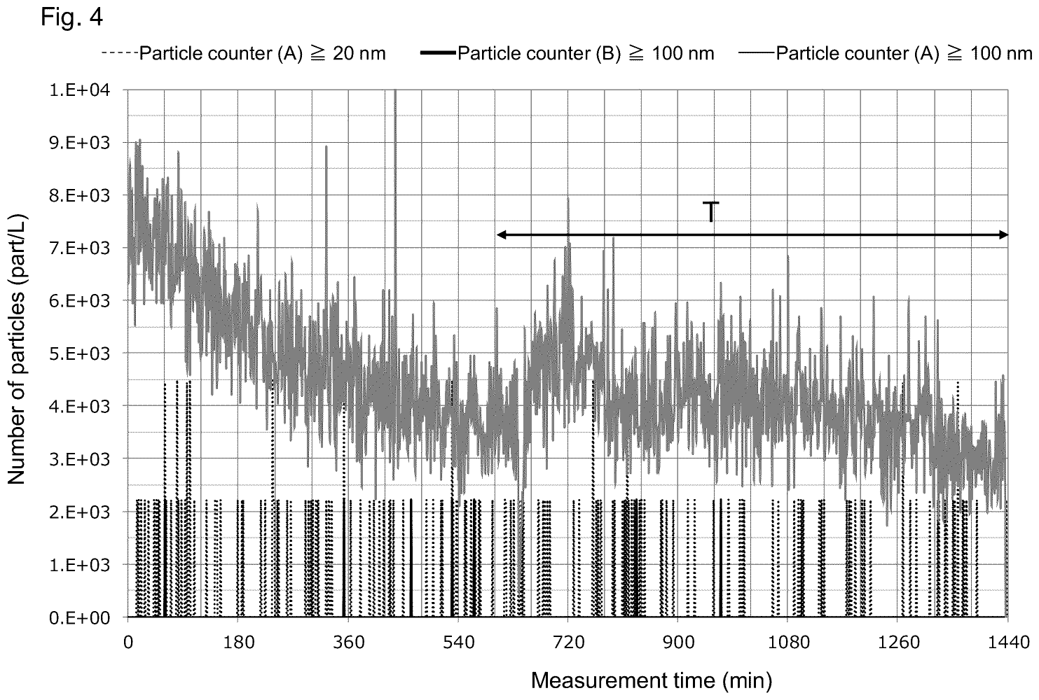

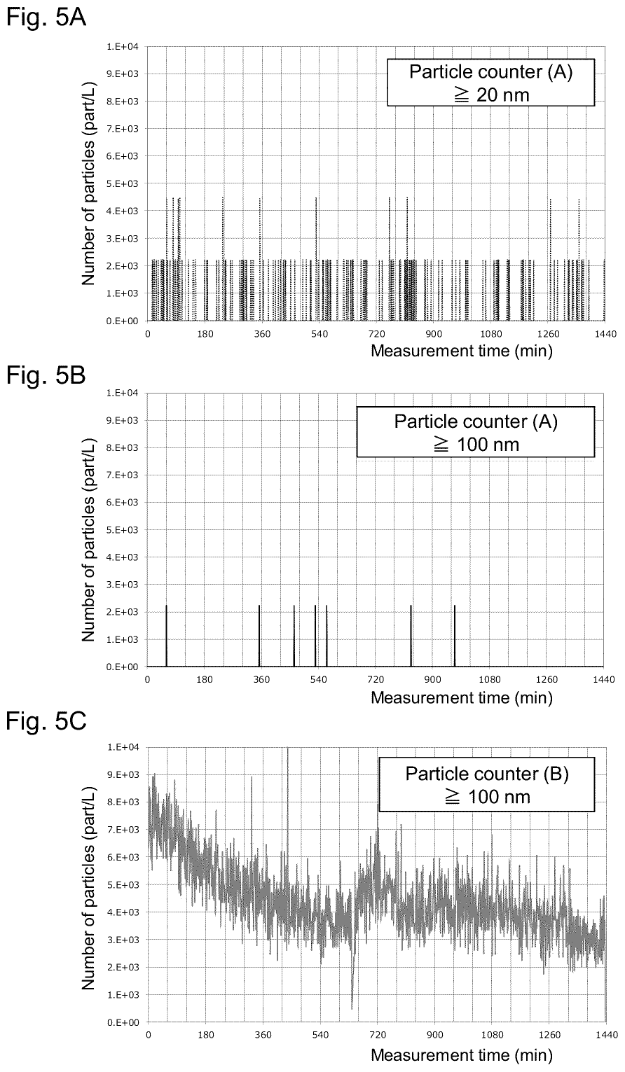

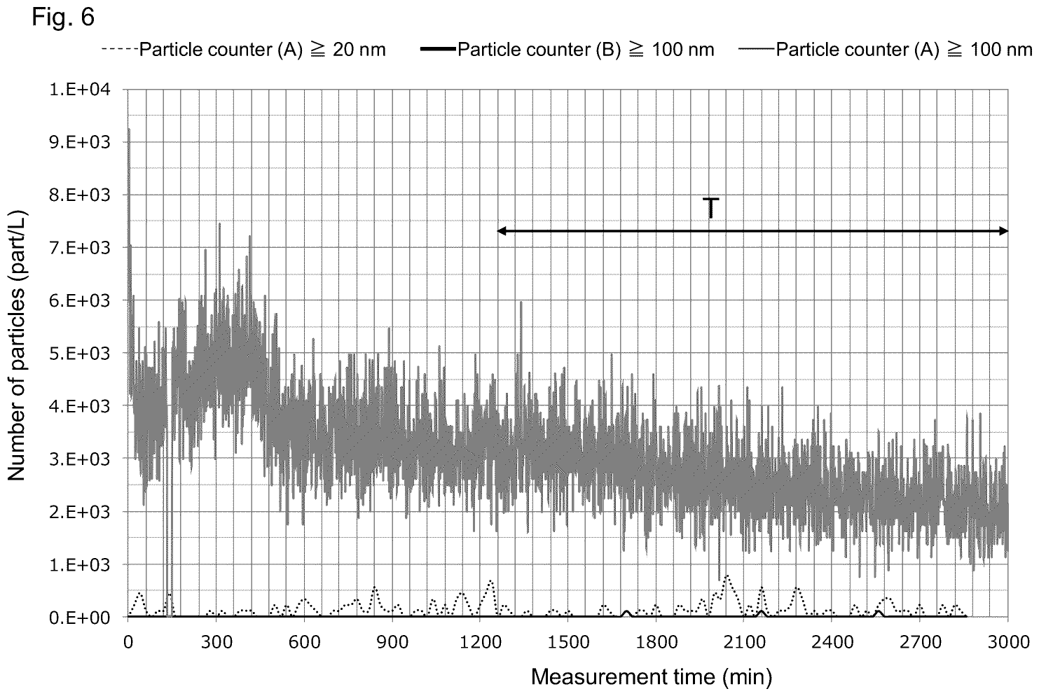

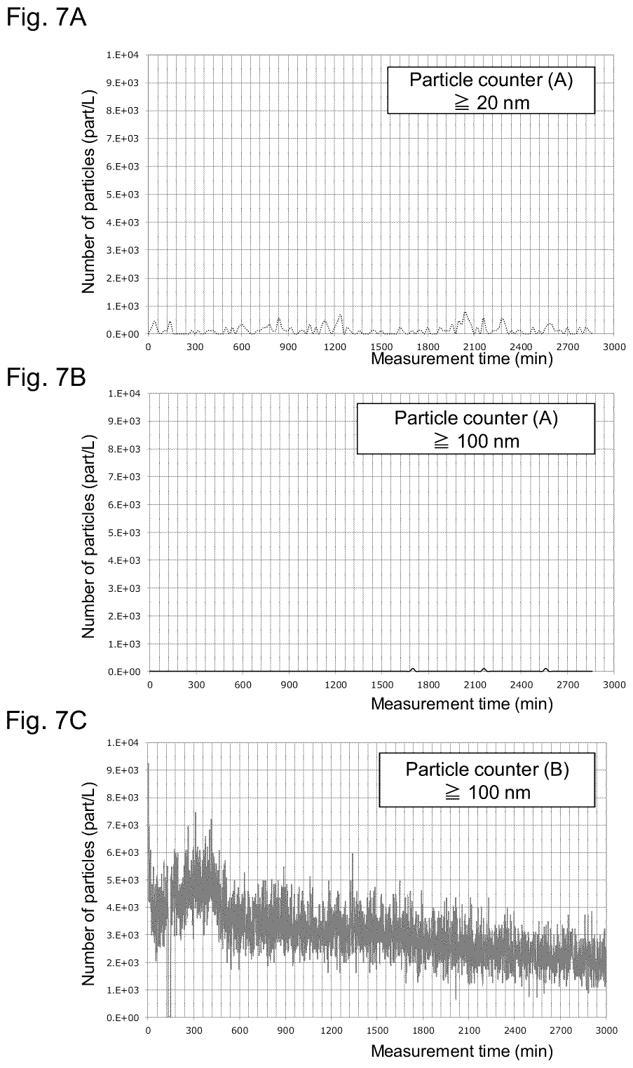

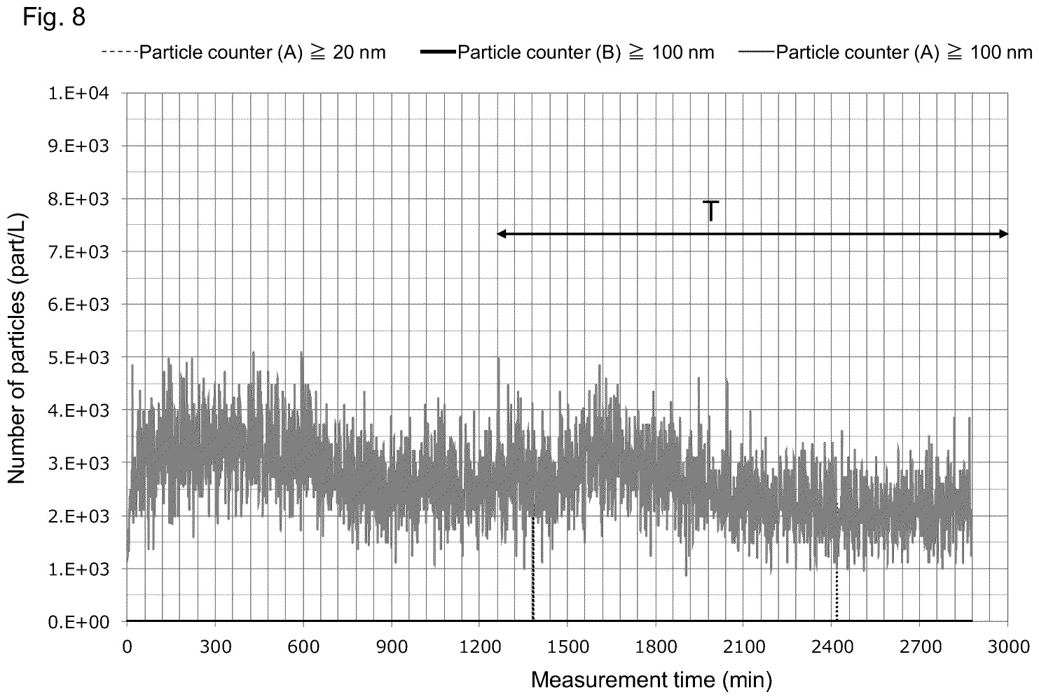

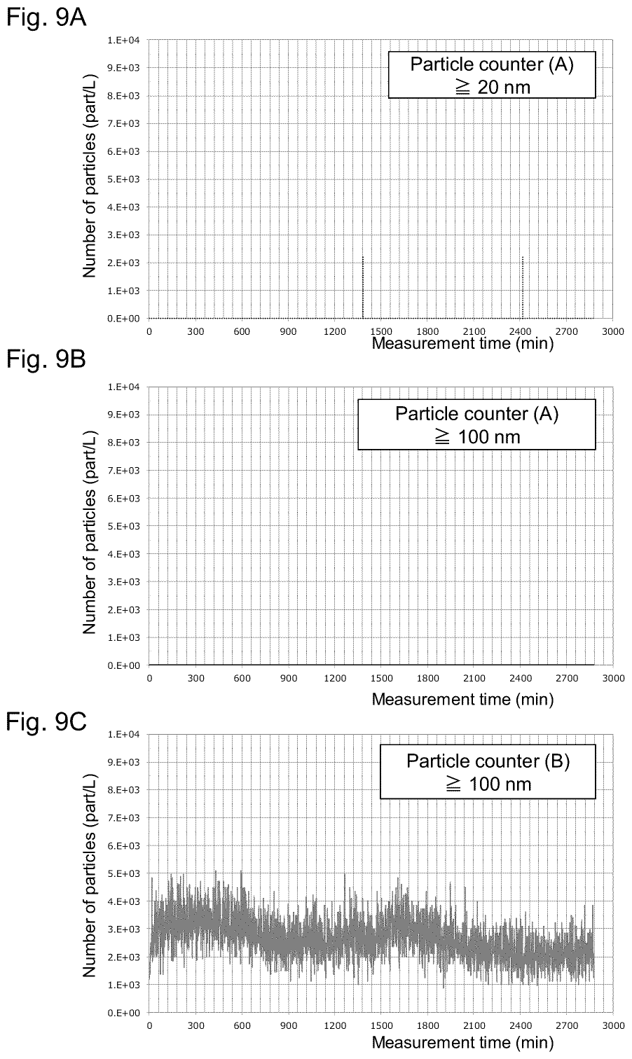

FIG. 4 shows the measurement results of particle counter (A) and particle counter (B) at measurement point P1 (i.e., shows the relationship between time and particle number). Particle counter (A) measured the particle number for 20 nm or more and the particle number for 100 nm or more. FIGS. 5A to 5C separately show the measurement results of the graph of FIG. 4, FIG. 5A showing the measurement results of the particle number for 20 nm or more realized by particle counter (A), FIG. 5B showing the measurement results of the particle number for 100 nm or more realized by particle counter (A), and FIG. 5C showing the measurement results of the particle number for 100 nm or more made by particle counter (B). Similarly, FIG. 6 shows the measurement results of particle counter (A) and particle counter (B) at measurement point P2. FIGS. 7A to 7C separately show the measurement results of the graph of FIG. 6 and are made in the same manner as FIGS. 5A to 5C. Similarly, FIG. 8 shows the measurement results of particle counter (A) and particle counter (B) at measurement point P3. FIGS. 9A to 9C separately show the measurement results of the graph of FIG. 8 and are made in the same manner as FIGS. 5A to 5C. Table 2 shows the average value of the measurements after the particle number was stabilized (the average value for time duration T shown in FIGS. 4, 6, and 8). In the SEM method, after confirming that the particle number was stabilized (the same state as in time duration T in FIGS. 4, 6, and 8), the water was supplied for a certain time to a centrifugal filtration apparatus having a particle capturing membrane of a pore size of 10 nm, following which the particles were sampled and observed. It should be noted that “<50” in the table means that the particle number is too small to be distinguished from signal noise and is considered to be equal to or less than a spurious number.

TABLE 2

| Measurement | Particle | P1 | P2 | P3 |
|---|---|---|---|---|
| method | diameter range | (part/L) | (part/L) | (part/L) |
| Particle counter | >=20 nm | 246 | 156 | <50 |
| (A) | >=100 nm | <50 | <50 | <50 |
| Particle counter | >=100 nm | 3,935 | 2,298 | 2,260 |
| (B) | | | | |
| SEM method | >=10 nm | 27,200 | — | 22,000 |
| | >=100 nm | 16,500 | — | 7,700 |

As will be understood from FIGS. 4 to 9C and Table 2, the particle numbers that were detected were smaller at measurement point P3 than at measurement point P1, and this tendency was found both in particle counter (A) and particle counter (B). On the other hand, particles having particle diameters of 100 nm or more were rarely detected by particle counter (A) but were detected by particle counter (B) and the SEM method. Particles having particle diameters of 100 nm or more are considered to be particles that were generated by ultrafiltration membrane apparatus 10 itself and not particles that passed through ultrafiltration membrane apparatus 10. One possible reason why particles having particle diameters of 100 nm or more were rarely detected by particle counter (A) is that particle counter (A) has only a limited region in which the laser beam is radiated, that is, particle counter (A) has only a limited particle detection region, and particles having particle diameters of 100 nm or more outside the particle detection region were not detected. On the other hand, since the laser beam irradiates a large region in particle counter (B), particle counter (B) has a large particle detection region and is therefore believed to have detected a larger number of particles having particle diameters of 100 nm or more than particle counter (A). In addition, it was found from the measurement data at measurement points P1 and P3 that the measurement results of particle counter (B) correlated with the measurement results of the SEM method. Specifically, a larger number of particles having particle diameters of 100 nm or more were detected at measurement point P1 than at measurement point P3 in the SEM method, and the same tendency was obtained in the measurement results of particle counter (B). On the other hand, particles of 100 nm or more that were present at a low concentration were rarely detected by particle counter (A).

As will be understood from the examples, particle counter (A) tends to be able to detect particles having small particle diameters, but its measurement accuracy for particles having large particle diameters tends to be worse, and the accurate measurement of particle numbers of every particle diameter by only particle counter (A) is difficult to obtain. Thus, the SEM method must be used to accurately measure the number of large particles. In the SEM method, a filter membrane is used that has a smaller pore size than the particle diameters of particles to be measured, and particles having small particle diameters can therefore be detected. Further, the SEM method can determine the shape of the particles and elements that form the particles as well as the particle number. However, the SEM method requires lengthy sampling using a centrifugal filtration apparatus, and a longer time is required for the filtering as the diameters of particles to be measured decrease. For this reason, it is difficult to promptly comprehend variation in the particle number in ultrapure water.

Based on the knowledge obtained from the examples, the inventors have arrived at the idea of using different particle counters having different counting efficiencies to measure particles having large particle diameters and particles having small particle diameters. Specifically, particle counter (A) is advantageous for detecting small particles but its particle detection region is small, while particle counter (B) is disadvantageous for detecting small particles but its particle detection region is large. Thus, the number of particles having small particle diameters is measured by a particle counter such as particle counter (A) having a low counting efficiency and a small measurable particle diameter, and the number of particles having large particle diameters is measured by a particle counter such as particle counter (B) having a large measurable particle diameter and high counting efficiency. In this manner, measurement that conventionally used the SEM method can be conducted only by particle counters, whereby the accuracy in measuring particle numbers can be improved for all particle diameters and prompt measurement can be ensured.

For this reason, first particle counter 12A (corresponding to particle counter (A)) and second particle counter 12B (corresponding to particle counter (B)) of particle measuring apparatus 12 have different counting efficiencies. Second particle counter 12B has higher counting efficiency than first particle counter 12A. The counting efficiencies of first particle counter 12A and second particle counter 12B are not limited, but because the counting efficiency is negatively correlated to the measurable particle diameter, the counting efficiency of first particle counter 12A is preferably selected from, for example, 10% or less, 5% or less, and 1% or less depending on the required measurable particle diameter. Second particle counter 12B is advantageous in that it has a high particle counting efficiency, and the counting efficiency is therefore preferably 50% or more, more preferably 60% or more, and still more preferably 70% or more. Similarly, the measurable particle diameters of first particle counter 12A and second particle counter 12B are not limited, but, for example, the measurable particle diameter of first particle counter 12A is preferably 50 nm or less and more preferably 20 nm or less. The measurable particle diameter of second particle counter 12B is preferably 100 nm or more.

Calculation means 12C calculates the distribution of particle numbers for all particle diameters by using the measurement results of first particle counter 12A for the particle numbers of particle diameters of less than 100 nm and the measurement results of second particle counter 12B for the particle numbers of particle diameters of 100 nm or more. Specifically, first particle counter 12A measures particle numbers for 20 nm or more, 50 nm or more, 75 nm or more, and 100 nm or more and can thereby measure particle numbers for 20 nm or more and less than 50 nm, 50 nm or more and less than 75 nm, 75 nm or more and less than 100 nm, and 100 nm or more. Regarding the particle number for 100 nm or more, the measurement results of second particle counter 12B is adopted instead of the measurement results of first particle counter 12A. In this manner, particle numbers for 20 nm or more and less than 50 nm, 50 nm or more and less than 75 nm, 75 nm or more and less than 100 nm, and 100 nm or more can be accurately obtained using different particle counters. If the measurable particle diameters of first particle counter 12A and the measurable particle diameters of second particle counter 12B partially overlap with each other, the measurement results of second particle counter 12B (the particle counter having higher counting efficiency) are preferably adopted.

Ultrapure water production apparatus (subsystem 1) has control unit 12D that manages the operation of the ultrapure water production apparatus based on the measurement results of calculation means 12C of particle measuring apparatus 12. Control unit 12D receives information of the particle number for each particle diameter range that is calculated by particle number calculation means 12C and judges whether the particle number of at least one of the particle diameter ranges for which the particle numbers are calculated by particle number calculation means 12C exceeds a predetermined threshold. Upon judging that a particle number exceeds the predetermined threshold, control unit 12D generates a signal showing that the particle number exceeds the predetermined threshold. Based on this signal, control unit 12D performs operation management of the ultrapure water production apparatus such as stopping the supply of the ultrapure water from the ultrapure water production apparatus to point of use 21 or stopping the operation of the ultrapure water production apparatus. Alternatively, control unit 12D outputs an alarm indicating that a particle number exceeds the predetermined threshold to an output device (not illustrated). In the present embodiment, an arrangement has been described in which the ultrapure water production apparatus (subsystem 1) has control unit 12D, but alternatively, particle measuring apparatus 12 may have control unit 12D.

The present invention has been described by means of embodiments, but the present invention is not limited to the embodiments described above. According to modifications, a plurality of sets of first particle counters 12A and second particle counters 12B may be provided at a plurality of positions. For example, sets of first particle counter 12A and second particle counter 12B may be provided at the inlet and the outlet of ultrafiltration membrane apparatus 10. In this case, particle number calculation means 12C may be separately provided for each set, or only one particle number calculation means 12C may be provided to receive information (the particle numbers) from each set and to output or display the information for each set. Second particle counter 12B may be omitted because sections other than the outlet of ultrafiltration membrane apparatus 10 are not affected or are only slightly affected, if at all, by particles having large particle diameters that are generated by ultrafiltration membrane apparatus 10 itself.

Although certain preferred embodiments of the present invention have been shown and described in detail, it should be understood that various changes and modifications may be made without departing from the spirit or scope of the appended claims.

### LIST OF REFERENCE NUMERALS

1 subsystem
2 primary pure water tank
3 pure water supply pump
4 ultraviolet ray oxidization apparatus
5 hydrogen peroxide removing apparatus
6 first ion exchange apparatus
7 membrane deaerator apparatus

1. A particle measuring apparatus comprising:
first and second particle counters that obtain a number of particles that are contained in water that flows in a predetermined section of an ultrapure water production apparatus; and
particle number calculator that calculates the number of the particles that are contained in the water that flows in the predetermined section for each particle diameter range based on measurement results of the first and second particle counters, wherein
the first particle counter and the second particle counter have different counting efficiencies.

2. The particle measuring apparatus according to claim 1, wherein the section is between a most downstream membrane filtering apparatus that constitutes the ultrapure water production apparatus and the point of use.

3. The particle measuring apparatus according to claim 1, wherein the counting efficiency of the second particle counter is higher than the counting efficiency of the first particle counter.

4. The particle measuring apparatus according to claim 1, wherein the first and second particle counters are provided at equivalent positions in the predetermined section.

5. The particle measuring apparatus according to claim 1, wherein a measurable particle diameter of the second particle counter is 100 nm or more.

6. The particle measuring apparatus according to claim 1, wherein a measurable particle diameter of the first particle counter is 20 nm or less.

7. An ultrapure water production apparatus comprising:
the particle measuring apparatus according to claim 1; and
a most downstream membrane filtering apparatus that constitutes the ultrapure water production apparatus.

8. The ultrapure water production apparatus according to claim 7, further comprising a control unit, wherein when the number of particles of at least one of the particle diameter ranges exceeds a predetermined threshold, the control unit generates a signal that shows that the number of particles exceeds the predetermined threshold, wherein the number of particles is calculated by the particle number calculator of the particle measuring apparatus for each particle diameter range.

9. The ultrapure water production apparatus according to claim 8, wherein the control unit manages operation of the ultrapure water production apparatus based on the signal.

10. A particle measuring method comprising:
using first and second particle counters to measure a number of particles that are contained in water that flows in a predetermined section of an ultrapure water production apparatus; and
based on measurement results of the first and second particle counters, using particle number calculator to calculate the number of the particles for each particle diameter range that are contained in the water that flows in the predetermined section, wherein
the first particle counter and the second particle counter have different counting efficiencies.
